(19) United States
(12) Patent Application Publication
(10) Pub. No.: US 2001/0033849 A1
(43) Pub. Date: Oct. 25, 2001

(54) Cosmetic compositions having retarding action on the regrowth of superfluous hair

(76) Inventor: Francesco Di Pierro, Milano (IT)

Correspondence Address: PENNIE & EDMONDS LLP, 1667 K STREET NW, SUITE 1000, WASHINGTON, DC, 20006

(21) Appl. No.: 09/781,301

(22) Filed: Feb. 13, 2001

(30) Foreign Application Priority Data
Mar. 24, 2000 (IT) MI2000A000628

(51) Int. Cl. A61K 35/78; A61K 7/06

(52) U.S. Cl. 424/401; 424/727; 424/757; 424/769

(57) ABSTRACT

The present invention relates to cosmetic compositions having retarding action on the regrowth of superfluous hair, more particularly to cosmetic compositions containing fatty acids and antiandrogenic sterols from Serenoa (Serenoa repens) and/or from Cucurbita seeds (Cucurbita pepo).

### FIELD OF THE INVENTION

The present invention relates to cosmetic compositions having retarding action on the regrowth of superfluous hair, more particularly to cosmetic compositions containing a fatty acid or an antiandrogenic sterol from Serenoa (Serenoa repens) and/or from Cucurbita seeds (Cucurbita pepo).

### BACKGROUND OF THE INVENTION

In humans, the main characteristics concerning the development of hair (number, color, thickness, length, and distribution) involve the consequence of an action mediated by sexual hormones, which in turn are derived from genetic expression.

In Western Countries, as well as in other socioeconomically advanced Countries, hair of limbs and armpits, pubic hair which can come out of bathing suits, as well as hair which can, although rarely, grow around the mammary areola, is considered unaesthetic in women. The term “superfluous hair” indicates indeed the growth of hair in these areas. As such superfluous hair is of course unwanted, it can be subjected to epilation (removal of the whole hair, roots included, by means of wax or electrolysis) or depilation (removal of the hair starting from the surface of the skin by chemical destruction of keratin, blade or electric shaving, or repeated traumatism of hair).

All of these procedures are, however, at least partly traumatic to the skin. These procedures may induce both inflammation in the tissues directly underlying the treated skin and lesions in capillaries and peripheral small vessels. There is therefore the need for such consequences to be reduced as well as for the regrowth of superfluous hair to be retarded as much as possible.

### SUMMARY OF THE INVENTION

One aspect of the present invention relates to a superfluous-hair-growth-retarding cosmetic composition comprising an active ingredient a fatty acid or an antiandrogenic sterol from Serenoa or from Cucurbita seeds. Advantageously, the active ingredient(s) are present in a therapeutically or cosmetically effective amount sufficient to provide a retarding action on the growth of superfluous hair. In one preferred embodiment, the amount of the active ingredient(s) present are from about 0.1% to about 3% by weight.

The composition may further include anti-inflammatory or vasal protective agents, e.g., such as triterpenes from liquorice; saponins from a horse chestnut plant; triterpenes from Terminalia sericea; isobutylamides from Zanthoxylum bungeanum; gingkoflavoneglucosides and terpenes from Ginkgo; polyphenols from grapes seeds; anthocyanosides from bilberries; saponins from butcher's broom; or a mixture thereof. In one embodiment, each anti-inflammatory or vasal protective agent is present in an amount from about 0.1% to about 3% by weight.

In a preferred embodiment, the composition includes a Serenoa repens extract and at least two anti-inflammatory or vasal protective agents. In another preferred embodiment, the composition includes a Cucurbita pepo extract and at least two anti-inflammatory or vasal protective agents.

The composition may also further include a pharmaceutically or cosmetically acceptable carrier, excipient, or adjunct, preferably including a phospholipid, which can form a complex with the active ingredients, thereby improving absorption or effectiveness.

Advantageously, the composition is in the form of a liquid, a solid, or a semi-solid, e.g., such as a lotion, a milk, a solution, an emulsion, a cream, a paste, a gel, a foam, or a combination thereof.

The composition may also further include one or more conventional depilatory agents, e.g., such as a thiol derivative; thioglycolic or thiolactic acid, or an alkali or alkaline-earth metal salt thereof; ethanolamine thioglycate; aminoethanethiol; mercaptopropionic acid; thioglycic or thioacetic acid; barium sulfate; or a mixture thereof.

The composition may also further include a conventional depilation accelerator, preferably based on a compound containing a urea, a thiourea, or a biguanide, or a combination of such compounds.

Another aspect of the invention relates to a cosmetic treatment method, which includes applying to a region of the body having superfluous hair a cosmetically or therapeutically effective amount of any of the compositions detailed above. Advantageously, the therapeutically or cosmetically effective amount is sufficient to provide a retarding action on the growth of superfluous hair. In addition, these methods can involve applying from about 0.01 mL to about 0.1 mL of the composition per square centimeter of skin area.

### DETAILED DESCRIPTION OF THE PREFERRED EMBODIMENTS

One aspect of the present invention relates to cosmetic compositions having retarding action on the regrowth of superfluous hair. More particularly, the present invention relates to cosmetic compositions containing a fatty acid or an antiandrogenic sterol from Serenoa (Serenoa repens) and/or from Cucurbita seeds (Cucurbita pepo). The compositions of the invention can contain either said pure components or extracts containing those components, preferably the components being present in an amount from about 0.1% to about 3% by weight.

Another aspect of the present invention relates to cosmetic compositions further containing anti-inflammatory and/or vasal protective agents, preferably including at least one of: anti-inflammatory triterpenes from liquorice (Glycyrrhiza glabra); anti-edematous and anti-inflammatory saponins from horse chestnut (Aesculus hyppocastanum); triterpenes from Terminalia sericea; analgesic and anti-inflammatory isobutylamides from Zanthoxylum bungeanum (sin Alatum); vasal protective gingkoflavoneglucosides and terpenes from ginkgo (Ginkgo biloba); vasal protective polyphenols from grapes seeds (Vitis vinifera); vasal protective anthocyanosides from bilberries (Vaccinium myrtillus); and vasal protective saponins from butcher's broom (Ruscus aculeatus). These preferred compositions of the invention can contain either the above indicated pure components or extracts containing them, preferably in an amount from about 0.1% to about 3% by weight.

All the derivatives listed above can optionally be complexed with pharmaceutically acceptable carriers, adjuncts, or excipients, which may also be capable of improving the absorption and effectiveness of the derivatives, such complexes particularly including phospholipids.

The extracts or derivatives indicated above, as well as the processes for the preparation thereof, may be found in, inter alia, U.S. Pat. No. 5,547,673; International Publication No. WO 00/02570; and European Patent Nos. EP 0,283,713, EP 0,250,953, and EP 0,275,224, the disclosures of all of which are incorporated herein by express reference hereto.

These derivatives can provide anti-inflammatory, analgesic, vasal tonifying, anti-edematous, and/or tonifying activity, which may make them useful in the treatment of inflammatory conditions and vasculopathies, and in dealing with capillary fragility. “Retarding action,” as used herein pertaining to hair regrowth, means an action which induces a delay in hair growth rate, a decrease in the amount of growing hair, or both.

According to the present invention, the cosmetic compositions can be in the form of post-depilatory or post-epilatory compositions, i.e., to be used subsequently depilation or epilation, or they may be included in depilatory formulations.

“Depilatory formulations,” as used herein, means formulations capable of removing hair by the cleavage of keratin disulfide bonds, thereby destroying hair or weakening it to the extent that it can easily be removed (e.g., mechanically by means of a spatula, a sponge, or simply by washing with water). Conventional depilatory formulations can further contain as an active ingredient thiol derivatives, such as thioglycolic or thiolactic acids, or an alkali or alkaline-earth metal salt thereof; ethanolamine thioglycate; aminoethanethiol; mercaptopropionic acid; thioglycic or thioacetic acids; barium sulfate; or a mixture thereof.

“Post-depilatory compositions,” as used herein without reference to post-epilatory compositions, should be understood to refer to both post-depilatory and post-epilatory compositions, for the sake of simplicity.

The compositions of the invention can be suitably formulated as a liquid, e.g., such as a lotion, a milk, a solution, an emulsion, or the like, or a combination thereof, or as a solid or semi-solid, e.g., such as a cream, a paste, a gel, a foam, or the like, or a combination thereof. For this purpose, the compositions of the invention may also contain pharmaceutically and/or cosmetically acceptable carriers, excipients, or adjuncts conventionally used for the preparation of formulations. These formulations may include those for topical use, thus including, for example, viscosity agents, such as tragacanth, xanthan gum, gypsum, clay, magnesium oxide, talc, or a combination thereof; chelating agents, such as ethylenediaminotetraacetic acid or disodium edetate; emollients, such as cetyl and stearyl alcohols, liquid paraffin, glyceryl stearate, silicon oils, hydrocarbon oils and waxes, or a combination thereof; wetting agents, such as urea or 1,3-butylene glycol; emulsifiers, such as medium chain triglycerids, acrylic acid polymers, ethoxylated stearyl or cetyl alcohols, polyoxyethylene alkyl ethers, or a mixture thereof; absorption promoters, such as cyclodextrans and liposomes; antioxidants, such as ascorbyl palmitate, tocopherol; preservatives, such as glycerin, propylene glycol; stabilizing agents; or fragrances; or a mixture thereof.

Other compositions according to the invention, such as depilatory compositions, may further contain other conventional additives, for example, depilation accelerators, such as compounds based on ureas, thioureas and biguanides.

Another aspect of the present invention further relates to a method of cosmetic treatment to retard the growth of superfluous hair. The method includes applying a cosmetically effective amount of the compositions of the invention on the body area where unwanted hair grows. The amount of the composition to be applied and the frequency of application will vary depending on the area to treat and on the personal characteristics of the user. Generally, it can range from about 0.01 mL to about 0.1 mL of a composition per cm² of skin area, typically to be applied about 1 to about 3 times a day during initial treatment, subsequently reducing the frequency, e.g., to approximately one application on alternate days, depending on the results obtained or desired.

Tests were carried out on healthy female volunteers, of age ranging from about 22 to about 40 years, using: a) post-depilatory compositions according to the invention, for use after superfluous hair had been removed with conventional methods; b) conventional depilatory compositions, containing the compositions of the invention; c) conventional depilatory compositions, containing no compositions of the invention.

In the case of treatments with a) and b), hair regrowth was complete not before about 3 weeks, progressively decreasing with the progress of treatment; furthermore, regrown hair was appeared weakened. Conversely, in treatment with c), hair regrowth was complete within about 2 to about 3 weeks.

### EXAMPLES

The following examples are only representative of the methods and materials for use in cosmetic compositions according to the invention, and are not to be construed as limiting the scope of the invention in any way.

### Example 1

Post-Depilatory Gel Formulation

A formulation according to the invention was prepared containing 2% lipophilic extract of serenoa repens, 0.3% ruscogenins, and 0.5% zanthoxylum bungeanum extract.

100 g contain:

| | | |
|---|---|---|
| Serenoa repens* lipophilic extract | 2 | g |
| Ruscogenins | 0.3 | g |
| 20% zanthoxylum bungeanum** extract solution | 0.5 | g |
| (in water) | | |
| Ethanol | 20 | g |
| PEG-6-Caprylic/Capric glycerides (SOFTIGEN 767, | 15 | g |
| from Huls) | | |
| Propylene glycol | 10 | g |
| Oleth 20 (VOLPO 20, from Croda) | 5 | g |
| Dimethicone copolyol | 2.5 | g |
| Carbomer | 2 | g |
| Triethanolamine | 1 | g |
| Zinc ricinoleate (TEGODOR HY77, from Teco) | 0.2 | g |
| Menthol | 0.5 | g |
| Preservatives | 0.5 | g |

| | |
|---|---|
| Antioxidants | q.s. |
| Purified water | q.s. to 100 g |

*Prepared according to U.S. Pat. No. 5,547,673.
**Prepared according to International Publication No. WO 00/02570.

### Example 2

Post-Depilatory Oil-in-Water Emulsion Formulation

A formulation according to the invention was prepared containing 1% glycyrrhetinic acid complex with phospholipids, 2% serenoa repens, and 1% ruscogenins.

100 g contain:

| | | |
|---|---|---|
| Glycyrrhetinic acid complex with phospholipids* | 1 | g |
| Ruscogenins** | 1 | g |
| Serenoa repens purified extract | 2 | g |
| Glyceryl stearate (CUTINA GMS, from Henkel) | 7 | g |
| Polysorbate 60 (TWEEN 60, from ICI) | 5 | g |
| Propylene glycol | 5 | g |
| Octyldodecanol (EUTANOL G, from Henkel) | 4 | g |
| Wheat germ oil | 3 | g |
| PEG-S soy sterols (GENEROL 122 ES, from Henkel) | 2 | g |
| Dimethicone | 1 | g |
| Carbomer | 0.6 | g |
| Tocopherol | 0.2 | g |
| Ascorbyl Palmitate | 0.1 | g |
| 10% NaOH solution (in water) | 2 | g |
| Disodium EDTA | 0.1 | g |
| Zine ricinoleate (TEGODOR CW90, from Tego) | 0.2 | g |

| | |
|---|---|
| Preservatives | q.s. |
| Fragrance | q.s. |
| Purified water | q.s. to 100 g |

*Prepared according to European Patent No. EP 0283713.
**Prepared according to European Patent No. EP 0250953.

### Example 3

Post-Depilatory Oil-in-Water Emulsion Formulation

A formulation according to the invention was prepared containing 1.5% Sericoside complex with phospholipids, 2% Cucurbita pepo lipophilic extract, and 0.5% ruscogenins.

100 g contain:

| | | |
|---|---|---|
| Sericoside complex with phospholipids* | 1.5 | g |
| Cucurbita pepo lipophilic extract | 2 | g |
| Ruscogenins C | 0.5 | g |
| Glycerin | 30 | g |
| EMULGADE SE (combination of glyceryl stearate, | 7 | g |
| Ceteareth-20, Ceteareth-12, Cetearyl Alcohol, and Cetyl | | |
| Palmitate, from Henkel) | | |
| Ceteareth-20 (EUMULGIN B2, from Henkel) | 2 | g |
| Dimethicone copolyol (SF 1188, from General Electric) | 2 | g |
| Zinc Ricinoleate (TEGODOR CW9O, from Tego) | 0.2 | g |
| Disodium edetate | 0.1 | g |
| Tocopherol | 0.2 | g |
| Ascorbyl Palmitate | 0.1 | g |

| | |
|---|---|
| Preservatives | q.s. |
| Fragrance | q.s. |
| Purified water | q.s. to 100 g |

*Prepared according to European Patent No. EP 0283713.

### Example 4

Post-Depilatory Oil-in-Water Emulsion Formulation

A formulation according to the invention was prepared containing 1.5% ESCIN/j3-Sitosterol complex with phospholipids, 2% Cucurbita pepo lipophilic extract, and 1% Ruscus aculeatus extract.

100 g contain:

| | | |
|---|---|---|
| ESCIN/f3-sitosterol complex with phospholipids* | 1.5 | g |
| Ruscus dry extract | 1 | g |
| Cucurbita pepo lipophilic extract | 2 | g |
| Glycerin | 5 | g |
| Polyisoprene (SYNTESQUAL, from Vevy) | 2.5 | g |
| Isostearyl isostearate (from Gattefoss) | 2.5 | g |
| Cetyl Palmitate (CRODAMOL CP, from Croda) | 2 | g |
| Decyl Oleate (CETIOL V, from Henkel) | 2 | g |
| Cetearyl Isononanoate (CETIOL SN, from Henkel) | 2 | g |
| Polysorbate 80 (TWEEN 80, from ICI) | 1 | g |
| Zinc Ricinoleate (TEGODOR CW9O, from Tego) | 0.2 | g |
| Tocopherol | 0.2 | g |
| Ascorbyl palmitate | 0.1 | g |
| Disodium edetate | 0.1 | g |
| SEPIGEL 30S (containing Polyacrylamide, C13-14 | 2 | g |
| isoparaffin, and laureth-7, from Seppic) | | |

| | |
|---|---|
| Preservatives | q.s. |
| Fragrance | q.s. |
| Purified water | q.s. to 100 g |

*Prepared according to European Patent No. EP 0283713.

### Example 5

Post-Depilatory Oil-in-Water Emulsion Formulation

A formulation according to the invention was prepared containing 1.5% glycyrrhetinic acid complex with phospholipids, 0.5% Vitis vinifera procyanidolic oligomers complex with phospholipids, and 2% Serenoa repens lipophilic extract.

100 g contain:

| | | |
|---|---|---|
| Glycyrrhetinic acid complex with phospholipids* | 1.5 | g |
| Vitis vinifera procyanidolic oligomers complex with | 0.5 | g |
| phospholipids** | | |
| Serenoa repens lipophilic extract | 2 | g |
| Propylene glycol | 5 | g |
| Modified jojoba oil (CETIOL J600, from Henkel) | 5 | g |
| Cetyl Alcohol | 5 | g |
| Glyceryl stearate (Cutina GMS - Henkel) | 5 | g |
| Polysorbate 60 (Tween 60 - ICI) | 3 | g |
| Hectorite (and) hydroxyethylcellulose | 0.5 | g |
| (Betone LT - Rheox) | | |
| Dimethicone 350 | 0.5 | g |
| Zinc Ricinoleate (TEGODOR CW90, from Tego) | 0.2 | g |
| Disodium edetate | 0.1 | g |
| Tocopherol | 0.2 | g |
| Ascorbyl palmitate | 0.1 | g |
| Sodium Metabisulfite | 0.1 | g |
| Citric acid | 0.1 | g |

| | |
|---|---|
| Preservatives | q.s. |
| Fragrance | q.s. |
| Purified water | q.s. to 100 g |

*Prepared according to European Patent No. EP 0283713.
**Prepared according to European Patent No. EP0275224.

### Example 6

Post-Depilatory Alcoholic Lotion Formulation

A formulation according to the invention was prepared containing 0.1% 18/β-glycyrrhetinic acid, 1% Gingko biloba, and 0.5% Serenoa repens.

100 mL contains:

| | | |
|---|---|---|
| 18/β-glycyrrhetinic acid | 0.1 | g |
| Gingko biloba dry extract (containing 20% | 1 | g |
| saponins) | | |
| Serenoa repens purified extract | 0.5 | g |
| Cyclomethicone | 10 | g |
| PEG-40 Hydrogenated castor oil (CREMOPHOR RH40, | 1 | g |
| from BASF) | | |
| Zinc Ricinoleate (TEGODOR HY77, from Tego) | 0.2 | g |
| Butylated Hydroxytoluene | 0.05 | g |

| | |
|---|---|
| Fragrance | q.s. |
| Alcohol | q.s. to 100 mL |

### Example 7

Post-Depilatory Gel Formulation

A formulation according to the invention was prepared having 2% Serenoa repens lipophilic extract.

100 g contain:

| | | |
|---|---|---|
| Serenoa repens lipophilic extract | 2 | g |
| Ethanol | 20 | g |
| PEG-6-Caprylic/Capric Glycerides (SOFTIGEN 767, | 15 | g |
| from Huls) | | |
| Propylene glycol | 10 | g |
| Oleth 20 (VOLPO 20, from Croda) | 5 | g |
| Dimethicone copolyol | 2.5 | g |
| Carbomer | 2 | g |
| Triethanolamine | 1 | g |
| Zinc ricinoleate (TEGODOR HY77, from Teco) | 0.2 | g |
| Menthol | 0.5 | g |

| | |
|---|---|
| Preservatives | q.s. |
| Antioxidants | q.s. |
| Purified water | q.s. to 100 g |

### Example 8

Post-Depilatory Oil-in-Water Emulsion Formulation

A formulation according to the invention was prepared having 2% Cucurbita pepo lipophilic extract.

100 g contain:

| | | |
|---|---|---|
| Cucurbita pepo lipophilic extract | 2 | g |
| Glycerin | 30 | g |
| EMULGADE SE (combination of glyceryl stearate, | 7 | g |
| Ceteareth-20, Ceteareth-12, Cetearyl Alcohol, and Cetyl | | |
| Palmitate, from Henkel) | | |
| Ceteareth-20 (EUMULGIN B2, from Henkel) | 2 | g |
| Dimethicone copolyol (SF 1188, from General Electric) | 2 | g |
| Zinc Ricinoleate (TEGODOR CW9O, from Tego) | 0.2 | g |
| Disodium edetate | 0.1 | g |
| Tocopherol | 0.2 | g |
| Ascorbyl Palmitate | 0.1 | g |

| | |
|---|---|
| Preservatives | q.s. |
| Fragrance | q. s. |
| Purified water | q.s. to 100 g |

It is to be understood that the invention is not to be limited to the exact configuration as illustrated and described herein. For example, it should be apparent that a variety of materials would be suitable for use in the cosmetic compositions or cosmetic treatment method according to the Detailed Description of the Preferred Embodiments. Accordingly, all expedient modifications readily attainable by one of ordinary skill in the art from the disclosure set forth herein, or by routine experimentation therefrom, are deemed to be within the spirit and scope of the invention as defined by the appended claims.

What is claimed is:

1. A cosmetic composition comprising an active ingredient comprising a fatty acid or an antiandrogenic sterol from Serenoa or from Cucurbita seeds in a therapeutically effective amount sufficient to provide a retarding action on the growth of superfluous hair.

2. The composition of claim 1, further comprising anti-inflammatory or vasal protective agents.

3. The composition of claim 2, wherein the anti-inflammatory or vasal protective agents comprise at least one of: triterpenes from liquorice; saponins from a horse chestnut plant; triterpenes from Terminalia sericea; isobutylamides from Zanthoxylum bungeanum; gingkoflavoneglucosides and terpenes from Ginkgo; polyphenols from grapes seeds; anthocyanosides from bilberries; saponins from butcher's broom; or a mixture thereof.

4. The composition of claim 3, wherein each anti-inflammatory or vasal protective agent is present in an amount from about 0.1% to about 3% by weight.

5. The composition of claim 1, which comprises a Serenoa repens extract and at least two anti-inflammatory or vasal protective agents.

6. The composition of claim 1, wherein comprises a Cucurbita pepo extract and at least two anti-inflammatory or vasal protective agents.

7. The composition of claim 1, further comprising a pharmaceutically or cosmetically acceptable carrier, excipient, or adjunct, which can form a complex with the active ingredients, thereby improving absorption or effectiveness.

8. The composition of claim 7, wherein the carrier comprises a phospholipid.

9. The composition of claim 1, wherein the active ingredient(s) are present in an amount from about 0.1% to about 3% by weight.

10. The composition of claim 1, wherein the composition is in the form of a liquid, a solid, or a semi-solid.

11. The composition of claim 1, wherein the composition is in the form of a lotion, a milk, a solution, an emulsion, a cream, a paste, a gel, a foam, or a combination thereof.

12. The composition of claim 1, which further comprises one or more conventional depilatory agents.

13. The composition of claim 12, wherein the one or more conventional depilatory agents comprise a thiol derivative; thioglycolic or thiolactic acid, or an alkali or alkaline-earth metal salt thereof; ethanolamine thioglycate; aminoethanethiol; mercaptopropionic acid; thioglycic or thioacetic acid; barium sulfate; or a mixture thereof.

14. The composition of claim 1, which further comprises a conventional depilation accelerator.

15. The composition of claim 14, wherein the conventional depilation accelerator comprises a urea, a thiourea, or a biguanide, or a combination thereof.

16. A cosmetic treatment method, which comprises applying to a region of the body having superfluous hair a composition comprising an active ingredient comprising a fatty acid or an antiandrogenic sterol from Serenoa or from Cucurbita seeds in a therapeutically effective amount sufficient to provide a retarding action on the growth of superfluous hair.

17. The cosmetic treatment method of claim 16, wherein from about 0.01 mL to about 0.1 mL of the composition is applied per square centimeter of skin area.
